(19) United States
(12) Patent Application Publication
(10) Pub. No.: US 2013/0172240 A1
(43) Pub. Date: Jul. 4, 2013

(54) METHODS FOR TREATING HCV

(71) Applicant: ABBVIE INC., North Chicago, IL (US)

(72) Inventor: Mudra J. Kapoor, Highland Park, IL (US)

(73) Assignee: ABBVIE INC., North Chicago, IL (US)

(21) Appl. No.: 13/718,167

(22) Filed: Dec. 18, 2012

Related U.S. Application Data
(60) Provisional application No. 61/580,871, filed on Dec. 28, 2011.

(51) Int. Cl. A61K 38/07; A61K 45/06; A61K 31/7056

(52) U.S. Cl. CPC A61K 38/07; A61K 31/7056; A61K 45/06
USPC 514/4.3

(57) ABSTRACT

The present invention features interferon-free therapies for the treatment of HCV. The therapies comprise administering Compound I (or a pharmaceutically acceptable salt thereof) and another anti-HCV agent. Preferably, the therapies are both interferon- and ribavirin-free.

This application claims priority from U.S. Provisional Patent Application Ser. No. 61/580,871, filed Dec. 28, 2011, which is incorporated herein by reference.

### FIELD OF THE INVENTION

The present invention relates to interferon-free treatment for HCV.

### BACKGROUND

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new therapies to treat HCV infection.

### SUMMARY OF THE INVENTION

Dimethyl (2S,2′S)-1,1′-((2S,2′S)-2,2′-(4,4′-((2S,5S)-1-(4-tert-butylphenyl) pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate

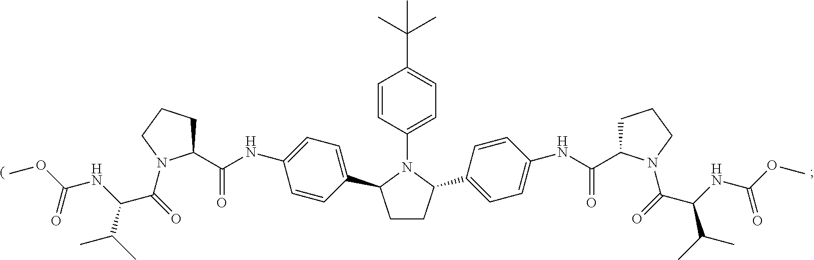

hereinafter “Compound I”) is a potent HCV NS5A inhibitor. Compound 1 is described in, for example, U.S. Publication No. 2010/0317568, which is incorporated herein by reference. The present invention features interferon-free therapies using Compound 1 (or a pharmaceutically acceptable salt thereof) in combination with one or more other HCV inhibitors.

In one aspect of the invention, the present invention features a method of treating an HCV patient, wherein the method comprises administering Compound I (or a pharmaceutically acceptable salt thereof), and one or more other anti-HCV agents, to the patient, and the treatment is interferon-free. The treatment may further comprise administering ribavirin to the patient. But the present invention also contemplates that the treatment can be ribavirin-free.

The other anti-HCV agent(s) that is combined or co-administered with Compound I (or the salt thereof) can be, for example and without limitation, an HCV protease inhibitor, an HCV polymerase inhibitor, an HCV NS5A inhibitor, an HCV entry inhibitor, a cyclophilin inhibitor, a CD81 inhibitor, or an internal ribosome entry site inhibitor. In one embodiment, the other anti-HCV agent(s) is an HCV polymerase inhibitor. In another embodiment, the other anti-HCV agent(s) is an HCV protease inhibitor.

In still another embodiment of this aspect of the invention, Compound I (or a pharmaceutically acceptable salt thereof) is combined or co-administered with two or more other anti-HCV agents. For instance, Compound I (or a pharmaceutically acceptable salt thereof) can be combined or co-administered with an HCV polymerase inhibitor and an HCV protease inhibitor. For another instance, Compound I (or a pharmaceutically acceptable salt thereof) can be combined or co-administered with two or more HCV protease inhibitors. For another instance, Compound I (or a pharmaceutically acceptable salt thereof) can be combined or co-administered with two or more HCV polymerase inhibitors (e.g., one is a nucleoside or nucleotide polymerase inhibitor and the other is a non-nucleoside polymerase inhibitor; or both are nucleoside or nucleotide polymerase inhibitors; or both are non-nucleoside polymerase inhibitor). In yet another example, Compound I (or a pharmaceutically acceptable salt thereof) is combined or co-administered with another HCV NS5A inhibitor and an HCV polymerase inhibitor. In still another example, Compound I (or a pharmaceutically acceptable salt thereof) is administered with two other different HCV NS5A inhibitors.

Compound I (or a pharmaceutically acceptable salt thereof) can be administered, for example and without limitation, concurrently with the other anti-HCV agent(s). Compound I (or a pharmaceutically acceptable salt thereof) can also be administered, for example and without limitation, sequentially with the other anti-HCV agent(s). For instance, Compound I (or a pharmaceutically acceptable salt thereof) can be administered immediately before or after the administration of the other anti-HCV agent(s). A short delay or time gap between the administration of Compound I (or a pharmaceutically acceptable salt thereof) and that of the other anti-HCV agent(s) is also contemplated.

Compound I (or a pharmaceutically acceptable salt thereof) can be co-formulated with the other anti-HCV agent(s) in a single dosage form. Non-limiting examples of suitable dosage forms include liquid or solid dosage forms. Preferably, the dosage form is a solid dosage form described in U.S. patent application Ser. No. 13/156,783, filed Jun. 9, 2011 and entitled ‘Solid Compositions”, the entire content of which is incorporated herein by reference. More preferably, the dosage form is a solid dosage form in which Compound I (or a pharmaceutically acceptable salt thereof) is molecularly dispersed in a matrix which comprises a pharmaceutically acceptable water-soluble polymer and a pharmaceutically acceptable surfactant. The other anti-HCV agent(s) can also be molecularly dispersed in the matrix, or formulated in different form(s).

As a non-limiting alternative, Compound I (or a pharmaceutically acceptable salt thereof) and the other anti-HCV agent(s) can be formulated in different dosage forms. For instance, Compound I (or a pharmaceutically acceptable salt thereof) and the other anti-HCV agent(s) can be formulated in different respective solid dosage forms.

A treatment according to the present invention can last for, for example and without limitation, 24 weeks or 48 weeks. Shorter treatment durations are also contemplated.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### DETAILED DESCRIPTION

Dimethyl (2S,2′S)-1,1′-((2S,2′S)-2,2′-(4,4′-((2S,5S)-1-(4-tert-butylphenyl)pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate

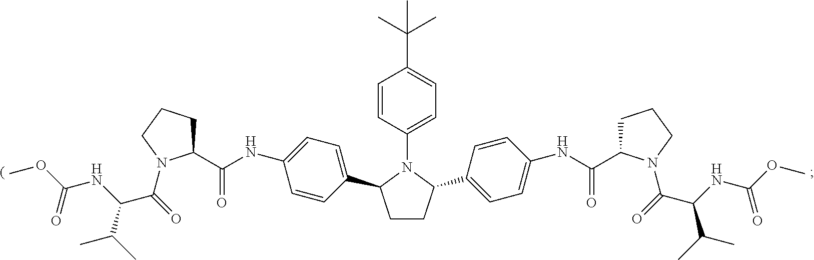

hereinafter “Compound I”) is a potent HCV NS5A inhibitor. The synthesis and formulation of Compound I are described in U.S. Publication No. 2010/0317568, and U.S. patent application Ser. No. 13/156,783 filed Jun. 9, 2011. All of these applications are incorporated herein by reference in their entireties.

The current standard of care for the treatment of HCV includes the use of pegylated interferon (e.g., pegylated interferon-alpha-2a or pegylated interferon-alpha-2b, such as Pegasys by Roche, or Peg-Intron by Schering-Plough) and the antiviral drug ribavirin (e.g., Copegus by Roche, Rebetol by Schering-Plough, or Ribasphere by Three Rivers Pharmaceuticals). The treatment often lasts for 24-48 weeks, depending on hepatitis C virus genotype. Other interferons include, but are not limited to, inferferon-alpha-2a (e.g., Roferon-A by Roche), interferon-alpha-2b (e.g., Intron-A by Schering-Plough), and interferon alfacon-1 (consensus interferon) (e.g., Infergen by Valeant).

The interferon/ribavirin-based treatment may be physically demanding, and can lead to temporary disability in some cases. A substantial proportion of patients will experience a panoply of side effects ranging from a “flu-like” syndrome (the most common, experienced for a few days after the weekly injection of interferon) to severe adverse events including anemia, cardiovascular events and psychiatric problems such as suicide or suicidal ideation. The latter are exacerbated by the general physiological stress experienced by the patients.

The present invention features the use of Compound I (or a pharmaceutically acceptable salt thereof) in combination with another anti-HCV agent to effectively treat HCV in an interferon-free therapy. Accordingly, in one aspect, the present invention features a method of treating an HCV patient, wherein the method comprises administering Compound I (or a pharmaceutically acceptable salt thereof), and one or more other anti-HCV agents, to the patient, and the treatment regimen does not include the use of interferon. In some cases, the treatment regimen does not include either interferon or ribavirin. In some other cases, the treatment regimen may further comprise administering ribavirin to the patient.

The other anti-HCV agent(s) that is co-administered with Compound I (or a pharmaceutically acceptable salt thereof) can be, for example and without limitation, an HCV protease inhibitor, an HCV polymerase inhibitor (e.g., a nucleoside polymerase inhibitor or a non-nucleoside polymerase inhibitor), an HCV helicase inhibitor, another HCV NSSA inhibitor, an HCV entry inhibitor, a cyclophilin inhibitor, a CD81 inhibitor, an internal ribosome entry site inhibitor, or a combination thereof. In one embodiment, the other anti-HCV agent(s) is an HCV polymerase inhibitor. In another embodiment, the other anti-HCV agent(s) is an HCV protease inhibitor.

In certain embodiments, the other anti-HCV agent(s) include two or more anti-HCV agents. For instance, the other anti-HCV agent(s) can include an HCV polymerase inhibitor and an HCV protease inhibitor. For another instance, the other anti-HCV agent(s) include two different HCV protease inhibitors. For another instance, the other anti-HCV agent(s) include two different HCV polymerase inhibitors (e.g., one is a nucleoside or nucleotide polymerase inhibitor and the other is a non-nucleoside polymerase inhibitor; or both are nucleoside or nucleotide polymerase inhibitors; or both are non-nucleoside polymerase inhibitor). In yet another example, the other anti-HCV agent(s) include another HCV NS5A inhibitor and an HCV polymerase inhibitor. In yet another example, the other anti-HCV agent(s) include another HCV NS5A inhibitor and an HCV protease inhibitor. In still another example, the other anti-HCV agent(s) include two other HCV NS5A inhibitors.

Specific examples of other anti-HCV agents that are suitable for the invention include, but are not limited to, PSI-7851 (Pharmasset), PSI-938 (Pharmasset), PF-00868554, ANA-598, IDX184, IDX102, IDX375, GS-9190, VCH-759, VCH-916, MK-3281, BCX-4678, MK-3281, VBY708, ANA598, GL59728, GL60667, BMS-790052, BMS-791325, BMS-650032, BMS-824393, GS-9132, ACH-1095, AP-H005, A-831 (Arrow Therapeutics), A-689 (Arrow Therapeutics), INX08189 (Inhibitex), AZD2836, telaprevir, boceprevir, ITMN-191 (Intermune/Roche), BI-201335, VBY-376, VX-500 (Vertex), PHX-B, ACH-1625, IDX136, IDX316, VX-813 (Vertex), SCH 900518 (Schering-Plough), TMC-435 (Tibotec), ITMN-191 (Intermune, Roche), MK-7009 (Merck), IDX-PI (Novartis), BI-201335 (Boehringer Ingelheim), R7128 (Roche), MK-3281 (Merck), MK-0608 (Merck), PF-868554 (Pfizer), PF-4878691 (Pfizer), IDX-184 (Novartis), IDX-375 (Pharmasset), PPI-461 (Presidio), BILB-1941 (Boehringer Ingelheim), GS-9190 (Gilead), BMS-790052 (BMS), CTS-1027 (Conatus), GS-9620 (Gilead), PF-4878691 (Pfizer), R05303253 (Roche), ALS-2200 (Alios BioPharma/Vertex), ALS-2158 (Alios BioPharma/Vertex), GSK62336805 (GlaxoSmithKline), or any combinations thereof.

Non-limiting examples of suitable HCV protease inhibitors include ACH-1095 (Achillion), ACH-1625 (Achillion), ACH-2684 (Achillion), AVL-181 (Avila), AVL-192 (Avila), BI-201335 (Boehringer Ingelheim), BMS-650032 (BMS), boceprevir, danoprevir, GS-9132 (Gilead), GS-9256 (Gilead), GS-9451 (Gilead), IDX-136 (Idenix), IDX-316 (Idenix), IDX-320 (Idenix), MK-5172 (Merck), narlaprevir, PHX-1766 (Phenomix), telaprevir, TMC-435 (Tibotec), vaniprevir, VBY708 (Virobay), VX-500 (Vertex), VX-813 (Vertex), VX-985 (Vertex), or a combination thereof. Non-limiting examples of suitable HCV polymerase inhibitors include ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), GS-6620 (Gilead), IDX-102 (Idenix), IDX-184 (Idenix), INX-189 (Inhibitex), MK-0608 (Merck), PSI-938 (Pharmasset), RG7128 (Roche), TMC64912 (Medivir), GSK625433 (GlaxoSmithKline), BCX-4678 (BioCryst), ALS-2200 (Alios BioPharma/Vertex), ALS-2158 (Alios BioPharma/Vertex), or a combination thereof A polymerase inhibitor may be a nucleotide polymerase inhibitor, such as GS-6620 (Gilead), IDX-102 (Idenix), IDX-184 (Idenix), INX-189 (Inhibitex), MK-0608 (Merck), PSI-938 (Pharmasset), RG7128 (Roche), TMC64912 (Medivir), ALS-2200 (Alios BioPharma/Vertex), ALS-2158 (Alios BioPharma/Vertex), or a combination therefore. A polymerase inhibitor may also be a non-nucleoside polymerase inhibitor, such as ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), or a combination thereof. Non-limiting examples of suitable NSSA inhibitors include GSK62336805 (GlaxoSmithKline), ACH-2928 (Achillion), AZD2836 (Astra-Zeneca), AZD7295 (Astra-Zeneca), BMS-790052 (BMS), BMS-824393 (BMS), GS-5885 (Gilead), PPI-1301 (Presidio), PPI-461 (Presidio), or a combination thereof. Non-limiting examples of suitable cyclophilin inhibitors include alisporovir (Novartis & Debiopharm), NM-811 (Novartis), SCY-635 (Scynexis), or a combination thereof. Non-limiting examples of suitable HCV entry inhibitors include ITX-4520 (iTherx), ITX-5061 (iTherx), or a combination thereof.

Compound I (or a pharmaceutically acceptable salt thereof) can be administered, for example and without limitation, concurrently with the other anti-HCV agent(s). Compound I (or a pharmaceutically acceptable salt thereof) can also be administered, for example and without limitation, sequentially with the other anti-HCV agent(s). For instance, Compound I (or a pharmaceutically acceptable salt thereof) can be administered immediately before or after the administration of the other anti-HCV agent(s). A short delay or time gap may exist between the administration of Compound I (or a pharmaceutically acceptable salt thereof) and that of the other anti-HCV agent(s). The frequency of administration may also be same or different. For example, Compound I (or a pharmaceutically acceptable salt thereof) may be administered once daily, and the other anti-HCV agent(s) may also be administered once daily. For another example, Compound I (or a pharmaceutically acceptable salt thereof) may be administered once daily, and the other anti-HCV agent(s) may be administered twice daily.

Compound I (or a pharmaceutically acceptable salt thereof) and the other anti-HCV agent(s) can be co-formulated in a single dosage form. Non-limiting examples of suitable dosage forms include liquid or solid dosage forms. Preferably, the dosage form is a solid dosage form described in U.S. patent application Ser. No. 13/156,783, filed Jun. 9, 2011 and entitled “Solid Compositions”, the entire content of which is incorporated herein by reference. More preferably, the dosage form is a solid dosage form in which Compound I (or a pharmaceutically acceptable salt thereof) is in an amorphous form, or highly preferably molecularly dispersed, in a matrix which comprises a pharmaceutically acceptable water-soluble polymer and a pharmaceutically acceptable surfactant. The other anti-HCV agent(s) can also be in an amorphous form or molecularly dispersed in the matrix, or formulated in different form(s) (e.g., in a crystalline form).

As a non-limiting alternative, Compound I (or a pharmaceutically acceptable salt thereof) and the other anti-HCV agent(s) can be formulated in different dosage forms. For instance, Compound I (or a pharmaceutically acceptable salt thereof) and the other anti-HCV agent(s) can be formulated in different respective solid dosage forms.

It will be understood that the total daily usage of the compounds and compositions to be administered will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily inhibitory dose of the compounds administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose.

In one embodiment, a treatment regimen of the invention comprises administering Compound I (or a pharmaceutically acceptable salt thereof), and one or more other anti-HCV agents, to an HCV patient, wherein the daily dose of Compound I (the salt thereof) is 5-300 mg. In another embodiment, a treatment regimen of the invention comprises administering Compound I (or a pharmaceutically acceptable salt thereof), and one or more other anti-HCV agents, to an HCV patient, wherein the daily dose of Compound I (the salt thereof) is 25 mg. Compound I (a pharmaceutically acceptable salt thereof) can be administered, without limitation, once daily.

A treatment regimen according to the present invention can last for, for example and without limitation, 20 weeks. Shorter treatment durations are also contemplated.

The following table lists non-limiting examples of the treatment regimens of the present invention. In each treatment regimen, Compound I (or a pharmaceutically acceptable salt thereof) and the other anti-HCV agent are administered daily to an HCV patient under such treatment. Each treatment is interferon-free. Administration of ribavirin can be included in each regimen. However, the present invention contemplates that each treatment regimen can be both interferon- and ribavirin-free. In addition, interferon and/or ribavirin can be included in the treatment regimen if needed. Each treatment regimen may also optionally comprise administering one or more other anti-HCV agents to the patient. The duration of each treatment regimen may depend on the patient's response. In any given regimen described below, the drugs can be, for example and without limitation, co-formulated in a single solid dosage form. For instance, all drugs used in a regimen can be co-formulated in amorphous forms or molecularly dispersed in a matrix comprising a water-soluble polymer and optionally a surfactant; for another instance, Compound I is formulated in an amorphous form or molecularly dispersed in a matrix comprising a water-soluble polymer and optionally a surfactant, and the other drug(s) are in crystalline form(s) and combined with amorphous Compound I in a single solid dosage form. For yet another instance, Compound I is formulated in a different dosage form than the other drug(s).

### Non-Limiting Examples of Interferon-free Treatment Regimens (with or without Ribavirin)

| Regimen | Drugs used in the treatment | |
|---|---|---|
| 1 | Compound I (or its salt) | ACH-1095 (Achillion) |
| 2 | Compound I (or its salt) | ACH-1625 (Achillion) |
| 3 | Compound I (or its salt) | ACH-2684 (Achillion) |
| 4 | Compound I (or its salt) | ACH-2928 (Achillion) |
| 5 | Compound I (or its salt) | alisporivir (Debio 025; Novartis) |
| 6 | Compound I (or its salt) | ALS-2158 |
| 7 | Compound I (or its salt) | ALS-2200 |
| 8 | Compound I (or its salt) | ANA-598 (setrobuvir, Anadys) |
| 9 | Compound I (or its salt) | ANA-773 (Anadys) |
| 10 | Compound I (or its salt) | AVL-181 (Avila) |
| 11 | Compound I (or its salt) | AVL-192 (Avila) |
| 12 | Compound I (or its salt) | AZD2836 (Astra-Zeneca) |
| 13 | Compound I (or its salt) | AZD7295 (Astra-Zeneca) |
| 14 | Compound I (or its salt) | BCX-4678 (BioCryst) |
| 15 | Compound I (or its salt) | BI-201335 (Boehringer |
| | | Ingelheim) |
| 16 | Compound I (or its salt) | BI-207127 (Boehringer |
| | | Ingelheim) |
| 17 | Compound I (or its salt) | BILB-1941 (Boehringer |
| | | Ingelheim) |
| 18 | Compound I (or its salt) | BMS-650032 (BMS) |
| 19 | Compound I (or its salt) | BMS-790052 (BMS) |
| 20 | Compound I (or its salt) | BMS-791325 (BMS) |
| 21 | Compound I (or its salt) | BMS-824393 (BMS) |
| 22 | Compound I (or its salt) | boceprevir |
| 23 | Compound I (or its salt) | CTS-1027 (Conatus) |
| 24 | Compound I (or its salt) | danoprevir |
| 25 | Compound I (or its salt) | VX-985 (Vertex) |
| 26 | Compound I (or its salt) | filibuvir (PF-00868554, Pfizer) |
| 27 | Compound I (or its salt) | GL59728 (Glaxo) |
| 28 | Compound I (or its salt) | GL60667 (Glaxo) |
| 29 | Compound I (or its salt) | GS-5885 (Gilead) |
| 30 | Compound I (or its salt) | GS-6620 (Gilead) |
| 31 | Compound I (or its salt) | GS-9132 (Gilead) |
| 32 | Compound I (or its salt) | GS-9256 (Gilead) |
| 33 | Compound I (or its salt) | GS-9451 (Gilead) |
| 34 | Compound I (or its salt) | GS-9620 (Gilead) |
| 35 | Compound I (or its salt) | GS-9669 (Gilead) |
| 36 | Compound I (or its salt) | GSK62336805 |
| 37 | Compound I (or its salt) | GSK625433 (GlaxoSmithKline) |
| 38 | Compound I (or its salt) | IDX-102 (Idenix) |
| 39 | Compound I (or its salt) | IDX-136 (Idenix) |
| 40 | Compound I (or its salt) | IDX-184 (Idenix) |
| 41 | Compound I (or its salt) | IDX-316 (Idenix) |
| 42 | Compound I (or its salt) | IDX-320 (Idenix) |
| 43 | Compound I (or its salt) | IDX-375 (Idenix) |
| 44 | Compound I (or its salt) | INX-189 (Inhibitex) |
| 45 | Compound I (or its salt) | ITX-4520 (iTherx) |
| 46 | Compound I (or its salt) | ITX-5061 (iTherx) |
| 47 | Compound I (or its salt) | MK-0608 (Merck) |
| 48 | Compound I (or its salt) | MK-3281 (Merck) |
| 45 | Compound I (or its salt) | MK-5172 (Merck) |
| 50 | Compound I (or its salt) | narlaprevir |
| 52 | Compound I (or its salt) | NM-811 (Novartis) |
| 53 | Compound I (or its salt) | PF-4878691 (Pfizer) |
| 54 | Compound I (or its salt) | PHX-1766 (Phenomix) |
| 55 | Compound I (or its salt) | PPI-1301 (Presidio) |
| 56 | Compound I (or its salt) | PPI-461 (Presidio--) |
| 57 | Compound I (or its salt) | Miravirsen (SPC3649; Santaris) |
| 58 | Compound I (or its salt) | PSI-938 (Pharmasset) |
| 59 | Compound I (or its salt) | mericitabine (RG7128; Roche) |
| 60 | Compound I (or its salt) | RO5303253 (Roche) |
| 61 | Compound I (or its salt) | SCY-635 (/Scynexis/) |
| 62 | Compound I (or its salt) | tegobuvir |
| 63 | Compound I (or its salt) | telaprevir |
| 64 | Compound I (or its salt) | TMC-435 (Tibotec) |
| 65 | Compound I (or its salt) | TMC-647055 (Tibotec) |
| 66 | Compound I (or its salt) | TMC64912 (Medivir) |
| 67 | Compound I (or its salt) | vaniprevir |
| 68 | Compound I (or its salt) | VBY708 (Virobay) |
| 69 | Compound I (or its salt) | VCH-759 (Vertex & ViraChem) |
| 70 | Compound I (or its salt) | VCH-916 (ViraChem) |
| 71 | Compound I (or its salt) | VX-222 (VCH-222) (Vertex & |
| | | ViraChem) |
| 72 | Compound I (or its salt) | VX-500 (Vertex) |
| 73 | Compound I (or its salt) | VX-759 (Vertex) |
| 74 | Compound I (or its salt) | VX-813 (Vertex) |
| 75 | Compound I (or its salt) | TMC649128 (Medivir) |
| 76 | Compound I (or its salt) | tegobuvir (GS-9190; Gilead) |
| 77 | Compound I (or its salt) | GI-5005 (GlobeImmune) |
| 78 | Compound I (or its salt) | IMO-2125 (Idera//) |
| 79 | Compound I (or its salt) | ITX-5061 (ITheRx) |
| 80 | Compound I (or its salt) | miR-122 (Regulus) |

It should be understood that the above-described embodiments and examples are given by way of illustration, not limitation. Various changes and modifications within the scope of the present invention will become apparent to those skilled in the art from the present description.

What is claimed is:

1. A method of treatment of an HCV patient, comprising administering to said patient Compound I or a pharmaceutically acceptable salt thereof, in combination of another anti-HCV agent, wherein said treatment is interferon-free.

2. The method of claim 1, wherein said treatment is ribavirin-free.

3. The method of claim 1, further comprises administering ribavirin to said patient.

4. A method according to one of claims 1-3, wherein said anti-HCV agent is a HCV polymerase inhibitor, an HCV protease inhibitor, an HCV entry inhibitor, a cyclophilin inhibitor, a CD81 inhibitor, or an internal ribosome entry site inhibitor.

5. A method according to one of claims 1-3, wherein said anti-HCV agent is an HCV polymerase inhibitor.

6. A method according to one of claims 1-3, wherein said anti-HCV agent is an HCV protease inhibitor.

7. A method according to one of claims 1-3, wherein said Compound I or salt thereof is administered concurrently with said another anti-HCV agent.

8. A method according to one of claims 1-3, wherein said Compound I or salt thereof is administered sequentially with said another anti-HCV agent.

9. A method according to one of claims 1-3, wherein said Compound I or salt thereof and said another anti-HCV agent are co-formulated in a solid dosage form.

10. A method according to one of claims 1-3, wherein said Compound I or salt thereof is formulated in a dosage form, and said another anti-HCV agent is formulated in another dosage form.
